SEQUENCE LISTING (35 sequences)

SEQ ID NO: 1 - DNA, 3713 bp, Homo sapiens
agcgccgctagcagcatgtctcggcgcaagcaggccaagccccagcacctcaagtcggacgaggagctgctgccgcctgacggggctcccgagcacgccgccccgggggaaggtgcggaggacgcagacagcgggcccgagagccgcagcgggggcgaggagaccagcgtgtgcgagaaatgctgcgccgagttcttcaagtgggcggacttcctggagcaccagcggagctgcaccaagctcccgcccgtgctgatcgtgcacgaggacgcgcccgcgccgccccccgaggacttccccgagccttcgcccgccagctcccccagcgagcgcgccgaaagcgaggcggccgaggaggcgggtgcggagggcgcggagggtgaggccaggccggtggagaaggaggccgagcccatggacgcggaacccgcgggggacacgcgcgcgccccggcccccgcctgcggcccctgcacccccaacgcccgcctacggcgcgcccagcaccaacgtgaccctggaggcgctgctgagcaccaaggtggcggtggcgcagttctcgcagggcgcgcgcgcggcaggcggctcgggagcaggtggaggcgtggcagctgcagccgtgcccctgatcctggaacagctcatggccctgcagcagcagcagatccaccagctgcagctcatcgagcagatccgcagccaggtggccctcatgcagcgcccgccgccgcggccctcactcagccccgcggccgccccgagcgcaccgggcccggcccccagccagctgcccgggctggccgcgctcccgctgtcggccggggcccctgccgccgccatcgcgggctcgggccccgccgccccggccgccttcgagggcgcgcagccgctgtcccggcccgagtctggcgccagcacccccggcggccctgcggagcccagcgcgcccgccgcccccagcgccgcccctgcccccgctgcccccgccccggcgccagcgccgcagagcgcggcctcgtcgcagccgcagagcgcatccacgccgcctgccctggccccggggtccctgctgggtgcggcgcccggcctgccaagtccgcttctacctcagacttccgccagcggcgtcatcttccccaacccgctggtcagcatcgcggccacggccaacgctctggacccgctgtccgcgctcatgaagcaccgcaagggcaagccgcccaatgtgtcggtgttcgagcccaaagccagcgccgaggacccgttcttcaagcacaaatgccgcttctgcgccaaggtcttcggcagcgacagcgcgctccagatccacctgcgctcgcacacaggcgagcggcccttcaagtgcaacatctgcgggaaccgcttctccaccaaaggcaacctgaaggtgcacttccagaggcacaaggagaagtacccccacatccagatgaacccttacccggtccccgagtacctggacaacgtgcccacctgctcgggcatcccctacggcatgtcgctgccccccgagaagcccgtgaccacctggctggacagcaagcccgtgctgcccaccgtgcccacgtccgtggggctgcaactgccgcccgctgtccctggcgcgcacggctacgccgactctcccagcgccaccccagccagccgctccccgcagaggccctcgcccgcctccagcgagtgcgcctccttgtccccaggcctcaaccacgtggagtccggcgtgtcggccaccgccgagtccccacagtcgctcctcggcgggccgcccgtcactaaagccgagcccgtcagcctgccctgcaccaacgccagggccggggacgctcccgtgggcgcgcaggctagcgctgcacccacatcggtggacggcgcacccacgagcctcggcagccccgggctgcccgccgtctccgagcagttcaaggcccagtttccgttcggggggctgctagactcgatgcaaacgtcggaaacctcgaagctgcagcagctggtggagaacatcgacaagaagatgacggacccgaaccagtgcgtcatctgccaccgggtgctgagctgccagagcgcgctgaagatgcactaccggacgcacacgggggagcggccgttcaagtgcaagatctgcggccgcgccttcaccaccaagggcaacctcaagacgcacttcggcgtgcaccgtgcaaagccgcccctgcgcgtgcagcactcctgccccatctgccagaagaagttcaccaacgccgtggtcctgcagcagcacatccgcatgcacatgggcggccagatccccaacacgccgctgccggagggcttccaggatgccatggactccgagctggcctacgacgacaagaacgcggagaccctgagcagctacgatgacgacatggacgagaactccatggaggacgacgctgagctgaaggacgcggccaccgacccggccaagccactcctgtcctatgcggggtcctgcccgccctccccgccctcggtcatctccagcattgccgccctggagaaccagatgaagatgatcgactcggtcatgagctgccagcagctgaccggcctcaagtccgtggagaacgggtccggggagagtgaccgcctgagcaacgactcctcgtcggccgtgggcgacctggagagccgcagcgcgggcagccccgccctgtccgagtcctcgtcctcgcaggccctgtcgccggcccccagcaatggtgagagcttccgctccaagtccccgggcctgggcgccccggaggagccccaggaaatcccgctcaagaccgagaggccggacagcccagccgccgccccgggcagcggaggcgcccctggccgcgcgggcatcaaggaggaggcgcccttcagcctgctgttcctgagcagggagcggggtcccagccaaagcactcctagcctgatctccagcgccgcacccaccatgatcaaaatggaagtgaacggtcacggcaaggccatggcgctgggcgagggtcccccgctgcccgcgggcgtccaggtccccgccgggcctcagacagtgatgggcccgggcctggcgcccatgctggcccccccaccgcgccggacgcccaagcagcacaactgccagtcgtgcgggaagaccttctcctcggccagcgccctgcagatccatgagcgcacgcacaccggcgagaagccgttcggctgcaccatctgcggccgggccttcaccactaagggcaacctcaaggtgcacatggggacacacatgtggaataacgcccccgcgagacgcggccgccgcctgtctgtggagaaccccatggctctcctagggggtgatgccctgaagttctctgaaatgttccagaaggacctggcagctcgggcaatgaacgtcgaccccagtttttggaaccagtatgctgcagccatcactaacgggctcgccatgaagaacaacgagatctccgtcatccagaacggcggcatcccccagctccccgtgagtcttgggggcagcgccctcccccctctgggcagcatggccagtgggatggacaaagcacgcactggcagtagcccacccatcgtcagcttggacaaagcgagctcagaaacagcagccagccgcccattcacgcggtttatcgaggataacaaggagattggtatcaactagccagtgactcg

SEQ ID NO: 2 - DNA, 2872 bp, Homo sapiens
gggcgctgggagacaccggacgcccgctcggctgcgctgcggctcaggcccccgctcgggcccgacccgctcggtcaccgccggctcgggcgcgcacctgccggctgcggccccagggccatgcggaggcccacgaggaggccggcggccacgcgcatcccgtagcccaggtggcccaggtctgcaccgcggcggcctcggcgccatggagcccccgtattcgctgacggcgcactacgatgagttccaagaggtcaagtacgtgagccgctgcggcgcggggggcgcgcgcggggcctccctgcccccgggcttcccgttgggcgctgcgcgcagcgccaccggggcccggtccgggctgccgcgctggaaccggcgcgaggtgtgcctgctgtcggggctggtgttcgccgccggcctctgcgccattctggcggctatgctggccctcaagtacctgggcccggtcgcggccggcggcggcgcctgtcccgagggctgccctgagcgcaaggccttcgcgcgcgccgctcgcttcctggccgccaacctggacgccagcatcgacccatgccaggacttctactcgttcgcctgcggcggttggctgcggcgccacgccatccccgacgacaagctcacctatggcaccatcgcggccatcggcgagcaaaacgaggagcgcctacggcgcctgctggcgcggcccgggggtgggcctggcggcgcggcccagcgcaaggtgcgcgccttcttccgctcgtgcctcgacatgcgcgagatcgagcgactgggcccgcgacccatgctagaggtcatcgaggactgcgggggctgggacctgggcggcgcggaggagcgtccgggggtcgcggcgcgatgggacctcaaccggctgctgtacaaggcgcagggcgtgtacagcgccgccgcgctcttctcgctcacggtcagcctggacgacaggaactcctcgcgctacgtcatccgcattgaccaggatgggctcaccctgccagagaggaccctgtacctcgctcaggatgaggacagtgagaagatcctggcagcatacagggtgttcatggagcgagtgctcagcctcctgggtgcagacgctgtggaacagaaggcccaagagatcctgcaagtggagcagcagctggccaacatcactgtgtcagagcatgacgacctacggcgagatgtcagctccatgtacaacaaggtgacgctggggcagctgcagaagatcaccccccacttgcggtggaagtggctgctagaccagatcttccaggaggacttctcagaggaagaggaggtggtgctgctggcgacagactacatgcagcaggtgtcgcagctcatccgctccacaccccaccgggtcctgcacaactacctggtgtggcgcgtggtggtggtcctgagtgaacacctgtccccgccattccgtgaggcactgcacgagctggcacaggagatggagggcagcgacaagccacaggagctggcccgggtctgcttgggccaggccaatcgccactttggcatggcgcttggcgccctctttgtacatgagcacttctcagctgccagcaaagccaaggtgcagcagctagtggaagacatcaagtacatcctgggccagcgcctggaggagctggactggatggacgccgagaccagggctgctgctcgggccaagctccagtacatgatggtgatggtcggctacccggacttcctgctgaaacccgatgctgtggacaaggagtatgagtttgaggtccatgagaagacctacttcaagaacatcttgaacagcatccgcttcagcatccagctctcagttaagaagattcggcaggaggtggacaagtccacgtggctgctccccccacaggcgctcaatgcctactatctacccaacaagaaccagatggtgttccccgcgggcatcctgcagcccaccctgtacgaccctgacttcccacagtctctcaactacgggggcatcggcaccatcattggacatgagctgacccacggctacgacgactgggggggccagtatgaccgctcagggaacctgctgcactggtggacggaggcctcctacagccgcttcctgcgaaaggctgagtgcatcgtccgtctctatgacaacttcactgtctacaaccagcgggtgaacgggaaacacacgcttggggagaacatcgcagatatgggcggcctcaagctggcctaccacgcctatcagaagtgggtgcgggagcacggcccagagcacccacttccccggctcaagtacacacatgaccagctcttcttcattgcctttgcccagaactggtgcatcaagcggcggtcgcagtccatctacctgcaggtgctgactgacaagcatgcccctgagcactacagggtgctgggcagtgtgtcccagtttgaggagtttggccgggctttccactgtcccaaggactcacccatgaaccctgcccacaagtgttccgtgtggtgagcctggctgcccgcctgcacgcccccactgcccccgcacgaatcacctcctgctggctaccggggcaggcatgcacccggtgccagccccgctctgggcaccacctgccttccagcccctccaggacccggtccccctgctgcccctcacttcaggaggggcctggagcagggtgaggctggactttggggggctgtgagggaaatatactggggtccccagattctgctctaagggggccagaccctctgccaggctggattgtacgggccccaccttcgctgtgttcttgctgcaaagtctggtcaataaatcactgcactgttaaaaaaaaaaaaa

SEQ ID NO: 3 - DNA, 3452 bp, Homo sapiens
atgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcggtgaaggacaaggaggagaaggacaggctgcacctcaaggagccgcccccgcccggccgccagcccccgcccgcgccgccggagcaggccgacggcaacgcgcccggtccgcagccgccgcccgtgcgcatccaggacatcaagaccgagaacggtacgtgcccctcgccgccccagcccctgtccccggccgccgccctgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacgaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcgagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttttgacacaccctcaaagccgaactaaatcgaaccccaaagcaggaaaagctaaaggaacccatcaaggcaaaatcgaaactaaaaaaaaaaaatccaattaaaaaaaacccctgagaatattcaccacaccagcgaacagaatatccctccaaaaattcagctcaccagcaccagcacgaagaaaactctattttcttaaccgattaattcagagccacctccactttgccttgtctaaataaacaaacccgtaaactgttttatacagagacagcaaaatcttggtttattaaaggacagtgttactccagataacacgtaagtttcttcttgcttttcagagacctgctttcccctcctcccgtctcccctctcttgccttcttccttgcctctcacctgtaagatattattttatcctatgttgaagggagggggaaagtccccgtttatgaaagtcgctttctttttattcatggacttgttttaaaatgtaaattgcaacatagtaatttatttttaatttgtagttggatgtcgtggaccaaacgccagaaagtgttcccaaaacctgacgttaaattgcctgaaactttaaattgtgctttttttctcattataaaaagggaaactgtattaatcttattctatcctcttttctttctttttgttgaacatattcattgtttgtttattaataaattaccattcagtttgaatgagacctatatgtctggatactttaatagagctttaattattacgaaaaaagatttcagagataaaacactagaagttacctattctccacctaaatctctgaaaaatggagaaaccctctgactagtccatgtcaaattttactaaaagtctttttgtttagatttattttcctgcagcatcttctgcaaaatgtactatatagtcagcttgctttgaggctagtaaaaagatatttttctaaacagattggagttggcatataaacaaatacgttttctcactaatgacagtccatgattcggaaattttaagcccatgaatcagccgcggtcttaccacggtgatgcctgtgtgccgagagatgggactgtgcggccagatatgcacagataaatatttggcttgtgtattccatataaaattgcagtgcatattatacatccctgtgagccagatgctgaatagatattttcctattatttcagtcctttataaaaggaaaaataaaccagtttttaaatgtatgtatataattctcccccatttacaatccttcatgtattacatagaaggattgcttttttaaaaatatactgcgggttggaaagggatatttaatctttgagaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagaggcaacacttaagcagtaattgctgttgcttgttgtcaaaatttgatcattgttaaaggattgctgcaaataaatacactttaatttcagtcaaaaa

SEQ ID NO: 4 - DNA, 2091 bp, Homo sapiens
atgagtgaaggggcagccgctgcctcgccacctggtgccgcttcggcagccgccgcctcggccgaggagggcaccgcggcggctgcggcggcggcagcggcgggcgggggcccggacggcggcggcgaaggggcggccgagcccccccgggagttacgctgtagcgactgcatcgtgtggaaccggcagcagacgtggctgtgcgtggtacctctgttcatcggcttcatcggcctggggctcagcctcatgcttctcaaatggatcgtggtgggctccgtcaaggagtacgtgcccaccgacctagtggactccaaggggatgggccaggaccccttcttcctctccaagcccagctctttccccaaggccatggagaccaccaccactaccacttccaccacgtcccccgccaccccctccgccgggggtgccgcctcctccaggacgcccaaccggattagcactcgcctgaccaccatcacgcgggcgcccactcgcttccccgggcaccgggtgcccatccgggccagcccgcgctccaccacagcacggaacactgcggcccctgcgacggtcccgtccaccacggccccgttcttcagtagcagcacgctgggctcccgacccccggtgccaggaactccaagtacccaggcaatgccctcctggcctactgcggcatacgctacctcctcctaccttcacgattctactccctcctggaccctgtctccctttcaggatgctgcctcctcttcttcctcttcttcctcctccgctaccaccaccacaccagaaactagcaccagccccaaatttcatacgacgacatattccacagagcgatccgagcacttcaaaccctgccgagacaaggaccttgcatactgtctcaatgatggcgagtgctttgtgatcgaaaccctgaccggatcccataaacactgtcggtgcaaagaaggctaccaaggagtccgttgtgatcaatttctgccgaaaactgattccatcttatcggatccaacagaccacttggggattgaattcatggagagtgaagaagtttatcaaaggcaggtgctgtcaatttcatgtatcatctttggaattgtcatcgtgggcatgttctgtgcagcattctacttcaaaagcaagaaacaagctaaacaaatccaagagcagctgaaagtgccacaaaatggtaaaagctacagtctcaaagcatccagcacaatggcaaagtcagagaacttggtgaagagccatgtccagctgcaaaattattcaaaggtggaaaggcatcctgtgactgcattggagaaaatgatggagtcaagttttgtcggcccccagtcattccctgaggtcccttctcctgacagaggaagccagtctgtcaaacaccacaggagtctatcctcttgctgcagcccagggcaaagaagtggcatgctccataggaatgccttcagaaggacacccccgtcaccccgaagtaggctaggtggaattgtgggaccagcatatcagcaactcgaagaatcaaggatcccagaccaggatacgataccttgccaagggtattcatccagtggtttaaaaacccaacgaaatacatcaataaatatgcaactgccttcaagagagacaaacccctattttaatagcttggagcaaaaggacctggtgggctattcatccacaagggccagttctgtgcccatcatcccttcagtgggtttagaggaaacctgcctgcaaatgccagggatttctgaagtcaaaagcatcaaatggtgcaaaaactcctattcagctgacgttgtcaatgtgagtattccagtcagcgattgtcttatagcagaacaacaagaagtgaaaatattgctagaaactgtccaggagcagatccgaattctgactgatgccagacggtcagaagactacgaactggccagcgtagaaaccgaggacagtgcaagcgaaaacacagcctttctccccctgagtcccacagccaaatcagaacgagaggcgcaatttgtcttaagaaatgaaatacaaagagactctgcattgaccaagtga

SEQ ID NO: 5 - DNA, 2608 bp, Homo sapiens
aaagggtggggggcgggggtagggggagccaatcccggcagcgccaaagaggggaggaggcggtgacagccgcggggagggggcgggaggagagaggcagctcggctcggctccgcgctcagctccgctctgcctccggctctgcgctcacctgctgcctagtgttccctctcctgctccaggacctccgggtagacctcagaccccgggcccattcccagactcagcctcagcccggacttccccagccccgacagcacagtaggccgccagggggcgccgtgtgagcgccctatcccggccacccggcgccccctcccacggcccgggcgggagcggggcgccgggggccatgcggggccagggccgcaaggagagtttgtccgattcccgagacctggacggctcctacgaccagctcacgggccaccctccagggcccactaaaaaagcgctgaagcagcgattcctcaagctgctgccgtgctgcgggccccaagccctgccctcagtcagtgaaattggccgggtcttccgctttctcggtgacagttcgctcccttcagcattagccgccccagcctccctccgcccccacagaccccgcctgctggacccagacagcgtggacgatgaatttgaattgtccaccgtgtgtcaccggcctgagggtctggagcagctgcaggagcaaaccaaattcacgcgcaaggagttgcaggtcctgtaccggggcttcaagaacgaatgtcccagcggaattgtcaatgaggagaacttcaagcagatttactcccagttctttcctcaaggagactccagcacctatgccacttttctcttcaatgcctttgacaccaaccatgatggctcggtcagttttgaggactttgtggctggtttgtccgtgattcttcggggaactgtagatgacaggcttaattgggccttcaacctgtatgaccttaacaaggacggctgcatcaccaaggaggaaatgcttgacatcatgaagtccatctatgacatgatgggcaagtacacgtaccctgcactccgggaggaggccccaagggaacacgtggagagcttcttccagaagatggacagaaacaaggatggtgtggtgaccattgaggaattcattgagtcttgtcaaaaggatgagaacatcatgaggtccatgcagctctttgacaatgtcatctagcccccaggagagggggtcagtgtttcctggggggaccatgctctaaccctagtccaggcggacctcacccttctcttcccaggtctatcctcatcctacgcctccctgggggctggagggatccaagagcttggggattcagtagtccagatctctggagctgaaggggccagagagtgggcagagtgcatctcggggggtgttcccaactcccaccagctctcacccccttcctgcctgacacccagtgttgagagtgcccctcctgtaggaattgagcggttccccacctcctacccctactctagaaacacactagacagatgtctcctgctatggtgcttcccccatccctgacctcataaacatttcccctaagactcccctctcagagagaatgctccattcttggcactggctggcttctcagaccagccattgagagccctgtgggagggggacaagaatgtatagggagaaatcttgggcctgagtcaatggataggtcctaggaggtggctggggttgagaatagaagggcctggacagattatgattgctcaggcataccaggttatagctccaagttccacaggtctgctaccacaggccatcaaaatataagtttccaggctttgcagaagaccttgtctccttagaaatgccccagaaattttccacaccctcctcggtatccatggagagcctggggccagatatctggctcatctctggcattgcttcctctccttccttcctgcatgtgttggtggtggttgtggtgggggaatgtggatgggggatgtcctggctgatgcctgccaaaatttcatcccaccctccttgcttatcgtccctgttttgagggctatgacttgagtttttgtttcccatgttctctatagacttgggaccttcctgaacttggggcctatcactccccacagtggatgccttagaagggagagggaaggagggaggcaggcatagcatctgaacccagtgtgggggcattcactagaatcttcaatcaacctgggctctccccaccccaccccagataacctcctcagttccctagggtctcttcttgcttgactcaatctacccagagatgccccttagcacacctagagggcagggaccataggacccaggttccaaccccattgtcagcaccccagccatgcggccaccccttagcacacctgctcgtcccatttagcttaccctcccagttggccagaatctgaggggagagcccccagagagcccccttccccatcagaagactgttgactgctttgcattttgggctcttctatatattttgtaaagtaagaaatataccagatctaataaaacacaatggctatgcaaaaaaaaaaaaaaaaaaaa

SEQ ID NO: 6 - DNA, 2100 bp, Homo sapiens
gtcccttctctctttgcctggaaagctttagctggataggctggaaaggcagcagggtcccaggggctctagggttgggcagaggcataatcacctccactttgtatagtgtttgcctcgtgcgaagtgcactccaccccacacttggccccacagcctcgctgtgcacctccttgcatcggccggacgggggcggggggtgcgggcggaggacggggaaggaggctcctgtgtcccctcttctcgcccctgcacacactcgcgccacggcgacccatgttgatcaccggagtttgggaagtcagaggggagccctctgctttcaggcctgggttccttctctaagatatgcccacttaggggcctgtgactcctccagaatgagtgtaccacactcgggacaccagaagcgagaggggacacaggctctgacctcgagagcacgtcagctctggagacccgcggccccagcctcgtgtgcctccgcacagggcagcttcagcactgcgaacagcgccagcaccgcggacagcgcgctcccacctcgcccccggaggccacgggctcgggccgtgttcagcaccgcggacagcgccagcaactccgagggtctcctggcctccagcccgccgtctctgacctccctgccccgccctcgactccttctcaaactttgggatgccccaggggaggaagagggcggcaggctgtgtgtggagtcttggcacggcgtacattacacagcccgatcaaaagccagcccgttcacctcgaccaagccagcatccaccaaagggccagaagtttaccaggccgctcaccgcgtgggcttccattctcctacctgcaagctgcgcgagttaaggagggaaagcacgtggcacagggcctggcttctggcgaggagcccaccagagatgcaaaggtgaggctgaaaccctttaaacttaaaaacaagaagatacaaacaattggaacaactggggacatgggtgctatgtggagggcaggacgcctggcttcccatcagggctgcagaggcggccgcggctggctccctctcccgggtgctgctggttgaacttggagagcaccctggctcctgagggtttaccgaccacagggtcaggccggggcagggattaccaccttacagatgcatagcctgaggccagtgaggacccacgctgtgctcagtgcccctggccggtaagtggtaggatttttatgtgaacgctgggttcctttcacacagcgtttttcatgtgcaaagtcataggtaattgtgtgactggtctctgagagaaagcatccaagcccagaggggtccttactcctcaggctctgtccctccagactctctcttattgcccctgccctctctggcctctgcctgccactggctacactgcatggggcagcagcatgcccaggttggagcctgcctgggatggccacattccctcccttgtccccaagctcctgctggtactcagggcttgaagcataggcattctcctgtctgtggctgccacagtcgtctaggggtctgtgtgggaatcaccagagtcatgttgcctggagacacatggtcaggagacccctcctgagtgggggtctgggggtgccttggccgcctggcctctgcctgccactgccccagactggctgcctcctgtctgctttccatcattctaaaatgtaaattgaatagtactcctccccttccagaagtgccagggaccatgtctaggctctgcagcctggtatcaggggccattcctggctggatccttccagctatcctgtatgctccctaggcctgtacccggtactccaagcccctccctgtgcctggatggccgagctgtcctctctgctggtggttactctgagcccctccctgtgcctggatggccgagccgccatgtcctctctgctggtggttactccgagcccctccctgtgcctggatggctgagctgccatgtcctctctgctggtggttactccgagcccctccctgtgcctggatggccgagctgccatgtcctctcggtgcccttagccactcactagtgcacatgcatcatccctgct

SEQ ID NO: 7 - DNA, 2501 bp, Homo sapiens
aaagtgtatatggctaatcctctcaagggcattccttcatgccttccctttgctctacaggacccagacagggcagggcatggagcaggcacacagtccgactgtctggctggactcctgacccttaccatttggtccaactgcattgtgctgagctcttgagccaccagtttatagggttttgacacagattatgccttgtctgtgtctttctctcactcgcccatccatccatccatccatccatccatccatccatccatccatgattaggaaagacatctatatttgttgagtacatgtttattaatgtgtagagaacacagcagagttggagtctcctaaaacatgacttgcactggctggtgccacctataggggttcaactgcttgattttctttttcttccttgtccctaccgccccccccgctcccccgccaccatccctctttgaataaatgaagatgttgaggttcttctatcgaggttccgctcactgatgctagatatctaaacttcatgcagagaagccatctacagaggcaacaggccagggtgggggcgagggggagggcctgaaagaagttgcccccttctgttgatctccgacccgcttgaaatctgaggcatctcctggggtcaggaggctgccttaatatccgcccccaacactaaacatttagcgcagggcaaggagaggcaggcgagggagggagggaaagggggaagcacatggccctgctttggcctgtttgcctcccataataagagggaaaggaatagaaaacacccaggcacaacataaatatttcctattatggagaaactaaagtcacaatggaatctgtcatgcttgtgatatgagagatggacaatatttcggggctgctgccatgctggcacctgaatggagtttatattttgtttatggagctgaatccgaagccttttatttttttggctcggcctgtgtcattcttcatggaagctcgaaatggccgcggagcagctcaatattccatttattttacttataaaagaaattcaaaggctgaaaagatgaagtaattgacttagtgggatgacagttaaacccactaaatttaatcttatcctctcaagtacaacatatcgcgcttacaaattatcgccagccaccaggcagccccgcgctcctcacctaatgcaaatggagcggaggcctctgttattgtattatctgactttactatcagcattaatccggcttagcccgcttaatgatgccacttgtggcactccgaaggcagcggagaagtctataaactacttccattaaaatgcaaatcgggcccgctgagaaaacaagtgacacaaaaagtgggcgccgcgcggccgccgcccgcgatggcgccgatggctgatgccgcgattacgccggcgggggcggcccgcgcgccacgaacgtgcccgacaagccgcgccgcttaatttctgaagagatttatattttttttgttgtcaggaaatgaacaaatacttatcaatgtaaaaactgctatctccgatctccccgccgggtttttcatactgatattctttgcaccccgtaacacaggaaatgaaaatcttctccgcctctcacgtcgccgctgcgccttcaaatcctctgcgagcagactggcctccccccaccggccccctcccgggctcccctccccggtaaacagaaaagaaaacttctgtgggcgaaggagggagaaattccaaggctctggctttccccagccgacttgggtttcggggcagcgcgggctcccttctttccagcgcaggaagaaggggtgagggaggaggggccggggtgctggcactcaacggtaacatctgttttacttaagcagctcgatttcgttttaggatgaaaaagtattccctttcagcatgaggaatcgtgggccctggggcttgtattgattcagaacaaggcctcataaaacactagcaggtgatttagtttcatattttgattcctttgtttagacagtttctctcaggttctcctgtgtcggcccggggaccccagcatccctcctttgatgttggagggacacccgggccgtcgagctcgccccgctcttgtgggtgactttgtgctcttgagggcgcagcatttcccaaggttctcccccaccctcacccggcgagcccaggctctgcggtcaggctctcaaatcctgcaaggattgatggtcctcacagagggatcaatactacattttaaatattacaataccttttattgagacactattacaacacaaatgttttaccctgtcacgggacccagttaaaattgcttttgaccatcttattggtgggcttccccggccctctgtgtttcccacttctgggaggagtcactgtgctcccgggatgggctggccccgcccaggaggcctgggaaggaggaggatgcagcctgggagggctgtggggcacctggggagtgctgcagaaa

SEQ ID NO: 8 - DNA, 24 bp, Artificial Sequence
  Description of Artificial Sequence Synthetic designed primer based on human gene
gttcgggttggtcgtttatcgttc

SEQ ID NO: 9 - DNA, 22 bp, Artificial Sequence
  Description of Artificial Sequence Synthetic designed primer based on human gene
cccacacactcgacccctaacg

SEQ ID NO: 10 - DNA, 21 bp, Artificial Sequence
  Description of Artificial Sequence Synthetic designed primer based on human gene
agacgtattggggcgaggggc

SEQ ID NO: 11 - DNA, 21 bp, Artificial Sequence
  Description of Artificial Sequence Synthetic designed primer based on human gene
ctccccgcgatcacacgcacg

SEQ ID NO: 12 - DNA, 25 bp, Artificial Sequence
  Description of Artificial Sequence Synthetic designed primer based on human gene
tttcgcggagacgttaatttagttc

SEQ ID NO: 13 - DNA, 24 bp, Artificial Sequence
  Description of Artificial Sequence Synthetic designed primer based on human gene
aaacgcaaaacgtatacaacgccg

SEQ ID NO: 14 - DNA, 24 bp, Artificial Sequence
  Description of Artificial Sequence Synthetic designed primer based on human gene
gagggttgggaaattgcggttttc

SEQ ID NO: 15 - DNA, 21 bp, Artificial Sequence
  Description of Artificial Sequence Synthetic designed primer based on human gene
ctcctcgcgctacgtcatccg

SEQ ID NO: 16 - DNA, 22 bp, Artificial Sequence
  Description of Artificial Sequence Synthetic designed primer based on human gene
gcgggtcggtattagttcggtc

SEQ ID NO: 17 - DNA, 25 bp, Artificial Sequence
  Description of Artificial Sequence Synthetic designed primer based on human gene
ctacgacgtataaaacccgtaaacg

SEQ ID NO: 18 - DNA, 24 bp, Artificial Sequence
  Description of Artificial Sequence Synthetic designed primer based on human gene
ggggttatgtaggcgcgttatttc

SEQ ID NO: 19 - DNA, 25 bp, Artificial Sequence
  Description of Artificial Sequence Synthetic designed primer based on human gene
tacgaatacacgctcataaaaaccg

SEQ ID NO: 20 - DNA, 20 bp, Artificial Sequence
  Description of Artificial Sequence Synthetic designed primer based on human gene
gggattcggccgtaggaggc

SEQ ID NO: 21 - DNA, 24 bp, Artificial Sequence
  Description of Artificial Sequence Synthetic designed primer based on human gene
caacgtaactcccgaaaaaactcg

SEQ ID NO: 22 - DNA, 24 bp, Artificial Sequence
  Description of Artificial Sequence Synthetic designed primer based on human gene
gtcgttttttgatcgatcggaggc

SEQ ID NO: 23 - DNA, 25 bp, Artificial Sequence
  Description of Artificial Sequence Synthetic designed primer based on human gene
aaaccgcgacaaaaacataaaaccg

SEQ ID NO: 24 - DNA, 26 bp, Artificial Sequence
  Description of Artificial Sequence Synthetic designed primer based on human gene
ttcgtttttcggttttattcgatgtc

SEQ ID NO: 25 - DNA, 26 bp, Artificial Sequence
  Description of Artificial Sequence Synthetic designed primer based on human gene
actaaacgaatctaaattaatccccg

SEQ ID NO: 26 - DNA, 26 bp, Artificial Sequence
  Description of Artificial Sequence Synthetic designed primer based on human gene
gatggttattttcgaggtttattagc

SEQ ID NO: 27 - DNA, 25 bp, Artificial Sequence
  Description of Artificial Sequence Synthetic designed primer based on human gene
cctccctttctaaaacgaaaatacg

SEQ ID NO: 28 - DNA, 29 bp, Artificial Sequence
  Description of Artificial Sequence Synthetic designed primer based on human gene
agtataatatatcgcgtttataaattatc

SEQ ID NO: 29 - DNA, 23 bp, Artificial Sequence
  Description of Artificial Sequence Synthetic designed primer based on human gene
aacgacgcgacttatcgaacacg

SEQ ID NO: 30 - DNA, 27 bp, Artificial Sequence
  Description of Artificial Sequence Synthetic designed primer based on human gene
gcgttttttatttaatgtaaatggagc

SEQ ID NO: 31 - DNA, 24 bp, Artificial Sequence
  Description of Artificial Sequence Synthetic designed primer based on human gene
taatcgcgacatcaaccatcgacg

SEQ ID NO: 32 - DNA, 25 bp, Artificial Sequence
  Description of Artificial Sequence Synthetic designed primer based on human gene
gagagtacgttagttttggagattc

SEQ ID NO: 33 - DNA, 24 bp, Artificial Sequence
  Description of Artificial Sequence Synthetic designed primer based on human gene
cacgtactttccctccttaactcg

SEQ ID NO: 34 - DNA, 26 bp, Artificial Sequence
  Description of Artificial Sequence Synthetic designed primer based on human gene
ttagtattgcgaatagcgttagtatc

SEQ ID NO: 35 - DNA, 29 bp, Artificial Sequence
  Description of Artificial Sequence Synthetic designed primer based on human gene
aataaatactaacttaatcgaaataaacg

(19) United States
(12) Patent Application Publication
(10) Pub. No.: US 2011/0218121 A1
(43) Pub. Date: Sep. 8, 2011

(54) GENE ASSOCIATED WITH LIVER CANCER, AND METHOD FOR DETERMINATION OF THE RISK OF ACQUIRING LIVER CANCER

(75) Inventors: Takeaki Ishizawa, Tokyo (JP); Norihiro Kokudo, Tokyo (JP); Hirohide Yoshikawa, Tokyo (JP); Yuko Shikauchi, Tokyo (JP); Yaeko Murase, Tokyo (JP)

(73) Assignee: THE UNIVERSITY OF TOKYO, Bunkyo-ku, TOKYO (JP)

(21) Appl. No.: 12/740,687

(22) PCT Filed: Oct. 29, 2008
(86) PCT No.: PCT/JP2008/003082

§ 371 Date: Jan. 21, 2011

(30) Foreign Application Priority Data
Oct. 30, 2007 (JP) 2007-281552

(51) Int. Cl. C40B 40/06; C12Q 1/68; C07H 21/04

(52) U.S. Cl. 506/16; 435/6.11; 536/24.31; 435/6.12

(57) ABSTRACT

Provided are a method for detecting early-stage liver cancer and determining a risk of liver cancer by using blood collected from a patient with imposing a less burden on the patient; and a gene marker, a probe, a primer, and a reagent kit that can be used in the detection. A marker for diagnosis of acquiring liver cancer includes a polynucleotide that can detect all of the following methylated genes and a gene region: (1) human SALL3c gene, (2) human ECEL1 gene, and (3) SEQ ID NO: 6 (NT_037622.5, 1393863-1395863).

### TECHNICAL FIELD

The present invention relates to a method for measuring a risk of acquiring liver cancer caused by a hepatitis virus or another cause. More specifically, the present invention relates to a method, a gene marker, and a diagnostic kit for determining the presence or absence of a genetic factor of liver cancer by detecting the presence or absence of methylation of a gene that affects a risk of liver cancer in a sample containing human genome DNA, such as a blood cell component or a plasma component collected from a subject. The invention also relates to a method for selecting a liver cancer therapeutic agent candidate.

### BACKGROUND ART

Liver cancer is a name referring to a malignant tumor occurring in the liver and can be roughly classified from its metastasis into a hepatocellular carcinoma, which primarily occurs in the liver cells, and metastatic liver cancer, which originates from an organ other than the liver and is transferred to the liver. A report of the Statistics Bureau in 1996 on the mortality from cancer shows that about 10000 patients die from liver cancer every year in Japan and that the mortality from liver cancer is 21.4% (1996), which is a major causative disease following stomach cancer, and, in 40s and 50s, the mortality from liver cancer is higher than that from stomach cancer.

Also, it has been recently reported that the number of deaths from liver cancer is 34000, which is about 11% of the total number of deaths from cancer. Ninety percent or more of the liver cancer patients are infected with hepatitis viruses, and it is also said that the number of viral hepatitis patients having a risk of developing liver cancer in Japan is three million. Hepatocellular carcinoma constitutes 95% of primary liver cancer, and the number of deaths therefrom reaches approximately 70% of all deaths from liver diseases. The causes of hepatocellular carcinoma are chronic hepatitis and liver cirrhosis due to hepatitis B and C, and the number of hepatocellular carcinoma cases is increasing each year. In about 70% of hepatitis C, the disease gradually progresses thereof and proceeds to liver cirrhosis and then liver cancer. It is known that approximately 80% of liver cancer cases are caused by hepatitis C.

Liver cancer is treated by resection of the liver, local treatment, hepatic artery therapeutic embolization, or radiotherapy. The resection of the liver is effective, provided that the liver function can endure the operation, and is most frequently employed. In the local treatment, a tumor is decreased in size by placing, for example, a needle into the tumor through the skin and injecting, for example, alcohol or is locally burned out by with a microwave or a radio wave. The hepatic artery therapeutic embolization kills cancer cells by occluding the hepatic artery to disconnect the cancer cells from their nutrient resource. Furthermore, the radiotherapy kills cancer cells by irradiating the focus as a target with a radiation beam. However, at present, the surgical operation is the most effective means.

It is highly possible to increase the survival rate of liver cancer patients by treatment at an early stage. In the case of hepatocellular carcinoma, it is reported that the five-year survival rate of early-stage hepatocellular carcinoma patients is 45%, whereas the five-year survival rate of non-early-stage hepatocellular carcinoma patients is 11%. In addition, the five-year survival rate of patients in clinical disease stage I is 91%, and those in stages II and III are 12% and 0%, respectively. Accordingly, early detection of liver cancer is important, and it has been desired to develop, for example, a diagnostic agent that can conveniently and highly accurately diagnose liver cancer. In addition, the risk of recurrence after operation of liver cancer is high, and diagnosis of the recurrence is also necessary.

At present, AST (GOT), ALT (GPT), and γ-GTP (γ-glutamine transpeptidase) are widely used as indicators of liver dysfunction. The tests are used for diagnosing liver dysfunction, but diagnosis of inflammation, fibrillation, and progress to cancer further requires virus testing. Furthermore, liver cancer is diagnosed, for example, by diagnostic imaging or using a tumor marker. As the diagnostic imaging that can diagnose the site progressing to liver cirrhosis or liver cancer, an abdominal ultrasound test, CT, or MRI is employed. The CT test and the ultrasound test can detect early cancer even if its size is 10 mm or less and exhibit high accuracy. However, the tests require insurance approval, a technical expert level, and highly equipped facilities, which are disadvantageous for frequently testing and for enabling any medical institution to conduct the tests. Also, as the markers of hepatocellular carcinoma, for example, α-fetoprotein (AFP) and protein induced by vitamin K absence of antagonist (PIVKA-2; des-gamma-carboxyprothrombin) are known.

However, AFP and PIVKA-2 have a disadvantage that they have a low positive rate of about 30 to 40%. A subject with an AFP level of 20 ng/mL or more is determined to have hepatocellular carcinoma. However, since AFP also increases in non-liver cancer patients, such as chronic hepatitis and active liver cirrhosis, discrimination among mild and moderate cases is difficult. Therefore, an AFP-L3 fraction, which is a tumor marker exhibiting higher specificity for hepatocellular carcinoma, is used in some cases. On the other hand, PIVKA-2 is a tumor marker having high specificity for hepatocellular carcinoma and rarely increases in other diseases. However, an increased level thereof is observed in, for example, alcoholic liver injury, during drug administration (for example, Warfarin or an antitubercular drug), and during vitamin shortage, despite not having hepatocellular carcinoma.

In the actual diagnosis at present, liver cancer is diagnosed using combination of AFP, AFP-L3 fraction, and PIVKA-2. Diagnosis of liver cancer using a blood marker is required to have an improved diagnostic yield for liver cancer. That is, an effective diagnostic marker having increased specificity and sensitivity is necessary.

Accordingly, a new method is desired to be developed, and, for example, a candidate thereof is the use of aberrant methylation of P16 gene, which is a cancer-related gene, as an indicator (Non-Patent Literature 1). Furthermore, proposed is a diagnostic method for determining a subject having severe hepatitis C progressing to liver cancer by measuring the GPC3 level in a sample of a hepatitis C patient (Patent Literature 1).
Non-Patent Literature 1: Wong I H N, et al., Detection of aberrant p16 methylation in the plasma and serum of liver cancer patients, Cancer Res., 59, 71-73, 1999.
Patent Literature 1: JP-A-2007-192557, Method for monitoring hepatitis C patient for progression to severe liver disease and liver cancer diagnostic method.

### SUMMARY OF INVENTION

### Technical Problem

However, conventional liver cancer markers or AFP and PIVKA-2 have disadvantages such that they are low in reactivity to early-stage cancer, that PIVKA-2 requires diagnostic equipment worth several million yen and needs time and effort because of combination with two or three types of other tumor markers, and that the type of tumor cannot be determined. Accordingly, an object to be solved by the present invention is to provide a convenient gene marker that has high reactivity also to early-stage cancer and high sensitivity for liver cancer and to provide a method for determining a risk of recurrence after resection of liver cancer. Furthermore, it is an object of the present invention to provide a method for searching a drug candidate that targets a methylated liver cancer marker gene.

### Solution to Problem

The present inventors have conducted intensive studies to solve the above-mentioned problems. As a result, the inventors have found the fact that methylated human SALL3c gene, human ECEL1 gene, human FOXC1 gene, human NRG3 gene, and human KCNIP2 gene, and methylated gene regions represented by SEQ ID NO: 6 (NT_{—}037622.5, 1393863 to 1395863) and SEQ ID NO: 7 (NT_{—}022135.15, 7774305 to 7776805) are liver cancer susceptible genes or gene regions. That is, it has been found that liver cancer can be detected by the presence or absence of methylation of these genes or gene regions or the frequency of the methylation. The present invention has proved that liver cancer can be detected using, as indicators, methylation of these genes or gene regions or a high frequency of the methylation, and has been thus accomplished. Furthermore, since a drug candidate that suppresses or prevents methylation of any of these genes has high possibility of becoming a liver cancer therapeutic drug, the present invention is also effective as a method for searching a drug candidate.

That is, the present invention provided the followings:

A method for detecting liver cancer by detecting the presence or absence of methylation of any one or more genes of human SALL3c gene, human ECEL1 gene, human FOXC1 gene, human NRG3 gene, and human KCNIP2 gene or detecting a frequency of the methylation;

A method for detecting liver cancer by detecting the presence or absence of methylation of either or both gene regions represented by SEQ ID NO: 6 (NT_{—}037622.5, 1393863 to 1395863) and SEQ ID NO: 7 (NT_{—}022135.15, 7774305 to 7776805) or detecting a frequency of the methylation;

A method for detecting liver cancer by detecting an expression level of human SALL3c gene, human ECEL1 gene, human FOXC1 gene, human NRG3 gene, or human KCNIP2 gene or an expression level of a gene region represented by SEQ ID NO: 6 (NT_{—}037622.5, 1393863 to 1395863) or SEQ ID NO: 7 (NT_{—}022135.15, 7774305 to 7776805), wherein the expression level to be detected includes an expression level of methylation of any of these genes and an expression level of any of these genes regardless of the presence or absence of methylation;

A method for detecting the presence or absence of methylation of human SALL3c gene, human ECEL1 gene, human FOXC1 gene, human NRG3 gene, or human KCNIP2 gene or a gene represented by SEQ ID NO: 6 (NT_{—}037622.5, 1393863 to 1395863) or SEQ ID NO: 7 (NT_{—}022135.15, 7774305 to 7776805) or detecting a frequency of the methylation, for detection of liver cancer, wherein a sample to be tested is, for example, either a plasma component or a blood cell component of a subject or the both;

A marker for diagnosis of acquiring liver cancer, the marker including a polynucleotide that can detect at least one of methylated genes or gene regions selected from the group consisting of the following seven: (1) human SALL3c gene, (2) human ECEL1 gene, (3) human FOXC1 gene, (4) human NRG3 gene, (5) human KCNIP2 gene, (6) a gene region represented by SEQ ID NO: 6 (NT_{—}037622.5, 1393863 to 1395863) and (7) a gene region represented by SEQ ID NO: 7 (NT_{—}022135.15, 7774305 to 7776805), wherein the marker for diagnosis of acquiring liver cancer including the polynucleotide are, for example, primers that can individually identify these methylated genes;

A marker for diagnosis of acquiring liver cancer, the marker including a polynucleotide that can detect all the following methylated genes and a gene region: (1) human SALL3c gene, (2) human ECEL1 gene, and (3) a gene region represented by SEQ ID NO: 6 (NT_{—}037622.5, 1393863 to 1395863), wherein the present invention provides a marker with higher sensitivity or a marker with higher specificity by detecting three methylated genes that have high detection rates of, in particular, liver cancer among the above-mentioned seven genes or gene regions found by the present inventors, and herein these genes may be configured into a microarray in which the polynucleotides are immobilized on a solid-phase surface;

A detection kit for detecting a risk, of recurrence after operation of liver cancer, the kit comprising the marker for diagnosis of acquiring liver cancer;

An oligonucleotide of human SALL3c gene represented by SEQ ID NO: 1, wherein the SALL3c gene is an alternate splice-form of SALL3 gene and has been newly found by the present inventors, who belong to Japanese Foundation For Cancer Research; and

A method for selecting a liver cancer therapeutic agent candidate, the method including the steps of culturing mammalian cells in the presence and absence of a test compound, measuring a frequency of methylation of human SALL3c gene, human ECEL1 gene, human FOXC1 gene, human NRG3 gene, human KCNIP2 gene, SEQ ID NO: 6, or SEQ ID NO: 7 in each cell, and selecting a test compound having an effect of suppressing the methylation as a liver cancer therapeutic agent candidate.

### Advantageous Effects of Invention

According to the present invention, provided is a new detecting method that can detect liver cancer at low cost, in particular, for example, early-stage liver cancer and a risk of recurrence after operation of liver cancer and that is very convenient and highly reliable. Early detection of liver cancer is a most effective method for decreasing the death rate from cancer, and a diagnostic marker and a method for detection at an early stage are desired. Furthermore, an oligonucleotide, a microarray, and a diagnostic kit that are used for the detecting method can be also provided. In addition, it is possible to search a drug candidate that suppresses or inhibits the methylation by using the methylation of the genes as an indicator.

### DESCRIPTION OF EMBODIMENTS

The present invention will be described in detail below. Note that embodiments are not limited to the followings.

The present invention provides a method for detecting liver cancer, in particular, early-stage liver cancer and recurrence of cancer after operation. The method is based on the finding that methylation of human SALL3c gene, human ECEL1 gene, human FOXC1 gene, human NRG3 gene, human KCNIP2 gene, or a gene represented by SEQ ID NO: 6 (NT_{—}037622.5, 1393863 to 1395863) or SEQ ID NO: 7 (NT_{—}022135.15, 7774305 to 7776805) and a high frequency of the methylation are indicators highly specific to liver cancer, and focuses on the methylation of these genes and the methylated genes or gene regions.

The method for detecting liver cancer by measuring methylation of a specific gene according to the present invention can be performed as follows: First, in the detecting method of liver cancer in the present invention, a serum, plasma, or blood cell component is collected from a subject. Examples of the sample to be tested include interstitial fluid, extravascular fluid, cerebrospinal fluid, synovial fluid, saliva, and liver tissue. A sample after operation is preferably collected from third day to seventh day after the operation. The tissue sample is, for example, tissue extracted by operation or with an endoscope or part of the tissue, a biopsy sample, or organ-washing liquid. Furthermore, the method for extracting DNA from these samples is well known in the field of the art, and, for example, extraction with phenol/chloroform can be employed. Alternatively, a commercially available DNA extraction reagent may be used. In such a case, a commercially available genomic DNA extraction kit and apparatus, such as GFX Genomic Blood DNA Purification Kit, may be used. Furthermore, a specific gene may be directly detected by, for example, PCR without through treatment for extracting DNA from a sample. In addition, in the detecting method of the present invention, only several milliliters of blood or about 10 mg of tissue in resection of cancer is sufficient as a sample. Thus, the method is a significantly less invasive and therefore imposes a less burden on patients or subjects.

The term “methylation of DNA” refers to that a cytosine (C), a base, of the DNA is methylated (addition of CH₃). The methylation is observed, in many cases, at a CpG site where C is situated next to G, and approximately 80% of CpG sites are methylated. A region on a genome where the density of CpG sites is high is called a CpG island and is thought to be involved in, for example, regulation of gene expression, cancer, and imprinting. In general, cytosines in the CpG islands are rarely methylated, and the CpG sites outside the CpG islands are methylated. In addition, a region of DNA in which a methylated CpG site is generated on only one DNA strand during DNA replication is returned to a state that both DNA strands are methylated by a function of methyltransferase. Therefore, methylated CpG sites and unmethylated sites are conserved even after the replication of DNA, and it is known to function as a marker on a genome. As biological meaning of the methylation, in particular, when a CpG island is present in the 5′ region of a gene, the methylation functions as an expression switch of the gene. Usually, the CpG island in the 5′ region of a gene is not methylated so that the gene can be expressed. For example, in cancer, the CpG island in the 5′ region of a tumor suppressor gene, which is usually unmethylated, is aberrantly methylated to suppress the expression, which has been recently recognized as an important oncogenic mechanism.

The term “nucleotide” refers to a nucleic acid. A polynucleotide is that nucleotides are joined to one another, includes DNA and RNA, and is the same meaning as a so-called primer, probe, or oligomer fragment.

In order to measure a degree or frequency of the methylation of DNA, for example, COBRA (combined bisulfite restriction analysis) or bisulfite sequencing can be used. These methods are based on a principle that unmethylated cytosine is converted to uracil, whereas methylated cytosine is not converted to uracil, by treating DNA with an unmethylated cytosine modifying reagent such as bisulfite. As technique for analyzing semi-quantitatively and with high sensitivity the methylation of DNA, Methylight is known. In the Methylight, methylation is detected by real-time PCR using combination of a primer recognizing a methylation specific sequence and a TaqMan probe. Furthermore, another example is a method using a restriction enzyme (such as Dnp1) that specifically cleaves a methylated gene sequence.

### Gene Sequence Information:

Information about genes that can be used in the method of the present invention is cited in the following Table 1 (SEQ ID NOs: 1 to 7).

Table 1 shows SEQ ID NOs., gene names, and Accession Nos. of genes that can be used in the present invention.

Any of the gene sequence information can be obtained using the Accession Nos. that can be confirmed in the website (http://www.ncbi.nlm.nih.gov/) of the National Biotechnology Information Center (NCBI). SALL3C, which does not have an Accession No., has been identified by the present inventors belonging to Japanese Foundation For Cancer Research as a new alternate splice form of SALL3 (NM_{—}171999) and is planed to be registered to a gene bank (NCBI). The gene sequence of SALL3C is shown as SEQ ID NO: 1. In the genes not named, the functions thereof are unclear whereas the gene sequence information is known. These genes not named are shown by the gene region positions that are used in the present invention.

TABLE 1

| | Gene Name | |
|---|---|---|
| SEQ ID NO: | (Gene Region Position) | Accession No. |
| SEQ ID NO: 1 | sal-like 3C (SALL3C) | SEQ ID NO: 1 |
| SEQ ID NO: 2 | Endothelin converting | NM_004826 |
| | enzyme-like I (ECEL1) | |
| SEQ ID NO: 3 | Forkhead box (FOXC1) | NM_001453 |
| SEQ ID NO: 4 | neuregulin 3 (NRG3) | NM_001010848 |
| SEQ ID NO: 5 | Kv channel interacting | NM_014591 |
| | protein 2 (KCNIP2) | |
| SEQ ID NO: 6 | 1393863 to 1395863 | NT_037622.5 |
| SEQ ID NO: 7 | 7774305 to 7776805 | NT_022135.15 |

### Detecting Method of Methylation in Gene According to the Present Invention:

A detecting method of methylation in a specific gene used in the detecting method of the present invention will be exemplarily shown below.

As technique for analyzing semi-quantitatively and with high sensitivity the methylation of DNA, Methylight and bisulfite sequencing are known. In the bisulfite sequencing, a single-chain DNA is treated with bisulfite (sodium hydrogen sulfite) to cause sulfonation and hydroamination. Subsequently, desulfonation is performed to convert cytosine to uracil. On the other hand, since the sulfonation rate of methylated cytosine is very slow, the methylated cytosine is not converted to uracil, but unmethylated cytosine is substituted by thymine (Frommer M, et al., Proc. Natl. Acad. Sci. USA, 891827-1831, (1992), Clark S J, et al., Nucleic Acids Res., 22, 2990 (1994)). The methylation status can be analyzed by utilizing the difference (C and T) in sequences. There are a number of methods utilizing bisulfite treatment for more specific experiments, such as bisulfite sequencing and combined bisulfite restriction analysis (COBRA), and a method can be selected from these methods so as to be suitable for the analysis purpose.

The DNA used for the detection of methylation is treated as follows: First, DNA is extracted from blood cells or serum using DNeasy Blood & Tissue kits (Qiagen). Subsequently, the DNA sample extracted from blood cells or serum is treated with bisulfite. A small amount of serum DNA or 500 ng of DNA extracted from blood cells or tissue is denatured by treatment with 0.2 M NaCl at 37° C. for 10 minutes. Then, sodium hydrogen sulfite in a final concentration of 3 M and hydroquinone in a final concentration of 0.5 mM are added thereto, followed by reaction for 16 hours. The reaction solution is desalted using Wizard DNA purification kit. The bisulfite treatment is performed in 0.3 M NaOH for 15 minutes and then is terminated. The modified DNA is precipitated with ethanol and then washed with 70% ethanol. The modified DNA is dissolved in 20 of distilled water. Alternatively, Epi TectBisulfite Kit (Qiagen) is used.

Then, primers for specifically detecting each of the methylated genes are prepared for detecting the presence or absence of methylation in the genes or gene regions shown in Table 1 or a frequency of the methylation according to the present invention. Table 2 shows the sequences (from 5′ to 3′) of sets of primers that can detect the respective genes used in Examples of the present invention. Then, nested PCR is performed using primers F1 and R1 in the first PCR and primers F2 and R2 in the second PCR. The nested PCR is a method for performing two-step PCR using external primers and internal primers. The first PCR products from an intended region are used as templates, and both primers are designed so as to recognize inner sides than the positions of the primers used first. The primer sequences are shown in Table 2, but are not limited thereto as long as they can specifically detect or identify the methylation of the genes. In the present invention, the marker for diagnosis of acquiring liver cancer may be primers that can amplify these specific genes. In order to amplify a gene sample by PCR, DNA polymerase having high fidelity is preferred. The primers are designed and synthesized using part of the gene sequence information shown in Table 2 in such a manner that the methylated specific gene as a target can be amplified. After the completion of the amplification reaction, the amplified products are detected for the presence or absence of methylation or a frequency of the methylation. In the present invention, the full length of each above-mentioned gene is not necessarily measured or detected, and a part of the gene may be measured or detected provided that each gene can be specified.

TABLE 2

| Gene SEQ ID NO: | |
|---|---|
| Gene Name, etc. | Primer Sequence |
| SEQ ID NO: 1 | F1: gttcgggttggtcgtttatcgttc (SEQ ID NO: 8) |
| SALL3C | R1: cccacacactcgacccctaacg (SEQ ID NO: 9) |
| | F2: agacgtattggggcgaggggc (SEQ ID NO: 10) |
| | R2: ctccccgcgatcacacgcacg (SEQ ID NO: 11) |
| SEQ ID NO: 2 | F1: tttcgcggagacgttaatttagttc (SEQ ID NO: 12) |
| ECEL1 | R1: aaacgcaaaacgtatacaacgccg (SEC) ID NO: 13) |
| | F2: gagggttgggaaattgcggttttc (SEQ ID NO: 14) |
| | R2: ctcctcgcgctacgtcatccg (SEQ ID NO: 15) |
| SEQ ID NO: 3 | F1: gcgggtcggtattagttcggtc (SEQ ID NO: 16) |
| FOXC1 | R1: ctacgacgtataaaacccgtaaacg (SEQ ID NO: 17) |
| | F2: ggggttatgtaggcgcgttatttc (SEQ.ID NO.: 18) |
| | R2: tacgaatacacgctcataaaaaccg (SEQ ID NO: 19) |
| SEQ ID NO: 4 | F1: gggattcggcgcgtaggaggc (SEQ ID NO: 20) |
| NRG3 | R1: caacgtaactcccgaaaaaactcg(SEQ ID NO: 21) |
| | F2: gtcgttttttgatcgatcggaggc (SEQ ID NO: 22) |
| | R2: aaaccgcgacaaaaacataaaaccg (SEQ ID NO: 23) |
| SEQ ID NO: 5 | F1: ttcgtttttcggttttattcgatgtc (SEQ ID NO: 24) |
| KCNIP2 | R1: actaaacgaatctaaattaatccccg (SEQ ID NO: 25) |
| | F2: gatggttattttcgaggtttattagc (SEQ ID NO: 26) |
| | R2: cctccctttctaaaacgaaaatacg (SEQ ID NO: 27) |
| SEQ ID NO: 6 | F1: agtataatatatcgcgtttataaattatc (SEQ ID NO: 28) |
| NT_037622.5 | R1: aacgacgcgacttatcgaacacg (SEQ ID NO: 29) |
| 1393863 to 1395863 | F2: gcgttttttatttaatgtaaatggagc (SEQ ID NO: 30) |
| | R2: taatcgcgacatcaaccatcgacg (SEQ ID NO: 31) |
| SEQ ID NO: 7 | F1: gagagtacgttagttttggagattc (SEQ ID NO: 32) |
| NT_022135.15 | R1: cacgtactttccctccttaactcg (SEQ ID NO: 33) |
| 7774305 to 7776805 | F2: ttagtattgcgaatagcgttagtatc (SEQ ID NO: 34) |
| | E2: aataaatactaacttaatcgaaataaacg (SEQ ID NO: 35) |

The gene amplification (PCR) in Examples of the present invention is performed as follows: Since a gene sample to be tested includes a large number of templates that cannot be amplified, Tth polymerase, which is a powerful tool, is used, and 2 μL of bisulfite-treated DNA is used as a template. The PCR reaction solution in the first PCR is prepared as shown in Table 3 using GeneAmp XL PCR Kit available from Applied Biosystems.

TABLE 3

| | |
|---|---|
| Template DNA | 2 μL |
| 3.3× XL buffer II | 15 μL |
| 25 mM Mg (OAc)₂ | 2.2 μL |
| 10 mM dNTP mix | 1 μL |
| primer A | 1 μL |
| primer B | 1 μL |
| rTth polymerase | 0.5 μL |
| Dw | 27.3 μL |

In the first step of PCR, a reaction cycle of denature at 94° C. for 30 seconds, annealing at 53 to 54° C. for 30 seconds, and extension at 68° C. for 3 minutes is repeated 40 times. Then, the PCR reaction solution for the second step of PCR is prepared as shown in Table 4 using GeneAmp XL PCR Kit available from Applied Biosystems. The PCR product in the first step is diluted with distilled water to 1/500 (in the case of serum DNA) or 1/1000 (in the case of blood cell DNA), and 1 μL of the diluted PCR product is used as the template DNA. In the second step of PCR, a reaction cycle of denature at 94° C. for 30 seconds, annealing at 53 to 54° C. for 30 seconds, and extension at 68° C. for 3 minutes is repeated 40 times.

TABLE 4

| | |
|---|---|
| Template DNA | 1 μL |
| 3.3× XL buffer II | 15 μL |
| 25 mM Mg (OAc)₂ | 2.2 μL |
| 10 mM dNTP mix | 1 μL |
| primer A | 1 μL |
| primer B | 1 μL |
| rTth polymerase | 0.5 μL |
| Dw | 28.3 μL |

### Kit for Diagnosis of Acquiring Liver Cancer:

In the present invention, the kit for diagnosis of acquiring liver cancer includes, in addition to the primers that can amplify each of the genes shown above, one or more components that are necessary for conducting the present invention. For example, the kit of the present invention can include a component for preserving or supplying an enzyme and a reaction component necessary for conducting the PCR. Examples of such components include, but not limited thereto, oligonucleotides of the present invention, enzyme buffer, dNTP, control reagents (such as target oligonucleotides for tissue samples and positive and negative controls), reagents for labeling or detection, a solid-phase support, and a manual.

### Method for Selecting Liver Cancer Therapeutic Agent Candidate:

The method for selecting a liver cancer therapeutic agent candidate includes the steps of culturing mammalian cells in the presence and absence of a test compound, measuring a frequency of methylation of human SALL3c gene, human ECEL1 gene, human FOXC1 gene, human NRG3 gene, human KCNIP2 gene, SEQ ID NO: 6 (NT_{—}037622.5, 1393863 to 1395863), or SEQ ID NO: 7 (NT_{—}022135.15, 7774305 to 7776805) in each cell, and selecting a test compound having an effect of suppressing the methylation as a liver cancer therapeutic agent candidate. The present invention is based on the finding that since the methylation of the genes and the gene regions is associated with liver cancer, a compound that can suppress or inhibit the methylation is identified as a liver cancer therapeutic agent candidate. Herein, cultured mammalian cells are used because methylation does not occur in yeast and Escherichia coli cells, or the physiological meaning of methylation differs from that of the present invention even if it occurs.

The present invention will be described more specifically with reference to Examples below, but is not limited thereto.

### Example 1

### Detection Rate in Each Stage of Liver Cancer of Each Gene Marker

Table 5 shows the results of a test of each gene marker for characteristics that can detect early-stage cancer in patients having liver cancer that is classified into well-differentiated (early-stage) cancer and moderately- or poorly-differentiated (advanced) cancer. All the patients are scheduled to be operated for resecting the liver cancer irrespective of sex. In addition, the classification to well-differentiation or moderately- or poorly-differentiation was accordance with the findings of medical doctors in charge of operation of the liver cancer. In order to evaluate the detection rates of liver cancer according to the present invention, detection rates of AFP and PIVKA-2, which are liver cancer markers conventionally used, were similarly investigated.

As shown in Table 5, though the AFP marker, which is conventionally used, showed low detection rates at every stages of liver cancer, the PIVKA-2 marker showed a high detection rate of 70% in well-differentiated cancer and a lower detection rate than the above of 44% in moderately- and poorly-differentiated cancer. The methylated SALL3C, ECEL1, and NT_{—}037622.5 (1393863 to 1395863) of the present invention showed high detection rates of 80%, 70%, and 80%, respectively, in well-differentiated cancer, which are higher than the detection rate of the PIVKA-2 marker and show their effectiveness. Here, the calculated detection rate is a ratio of the number of patients that reacted to a gene marker (a group in which methylated gene is detected) to the number of patients having well-differentiated cancer or moderately- or poorly-differentiated cancer. Furthermore, it is also possible to detect moderately- or poorly-differentiated cancer in consideration of that clinical research showing “any one of the markers is positive before operation and changes to negative after the operation” and that healthy individuals show negative in any of the genes in Example 3.

TABLE 5

| | | Moderately- or |
|---|---|---|
| SEQ ID NO:, Accession No., | | Poorly- |
| Gene Name | Well-differentiated | differentiated |
| (Gene Region Position) | Cancer | Cancer |
| SEQ ID NO: 1 | 8 subjects (80%) | 24 subjects (53%) |
| SALL3C | | |
| SEQ ID NO: 2, NM_004826 | 7 subjects (70%) | 17 subjects (38%) |
| ECEL1 | | |
| SEQ ID NO: 3, NM_001453 | 5 subjects (50%) | 10 subjects (22%) |
| FOXC1 | | |
| SEQ ID NO: 4, | 4 subjects (40%) | 8 subjects (18%) |
| NM_001010848 | | |
| NRG3 | | |
| SEQ ID NO: 5, NM_014591 | 2 subjects (20%) | 5 subjects (11%) |
| KCNIP2 | | |
| SEQ ID NO: 6, NT_037622.5 | 8 subjects (80%) | 28 subjects (62%) |
| 1393863 to 1395863 | | |
| SEQ ID NO: 7, NT_022135.15 | 4 subjects (40%) | 2 subjects (4%) |
| 7774305 to 7776805 | | |
| AFP | 2 subjects (20%) | 11 subjects (24%) |
| PIVKA II | 7 subjects (70%) | 20 subjects (44%) |

### Example 2

### Detection Rate in Vascular Invasion of Liver Cancer of Each Gene Marker

The vascular invasion of liver cancer means that cancer cells invade the circumference of the portal vein, the hepatic vein, the hepatic artery, and the bile duct or into these vascular systems, and includes macroscopic vascular invasion that can be diagnosed by image inspection or visual inspection of a resected specimen and microscopic vascular invasion that can be first diagnosed under a microscope. The microscopic vascular invasion is known as one of the most powerful prognostic factors against recurrence after treatment, but it is difficult for the current technology to diagnose before operation whether or not the microscopic vascular invasion is present. The results shown in Table 6 show the AFP marker and the PIVKA-2 marker, which are conventionally used, cannot determine the presence or absence of vascular invasion of liver cancer. On the other hand, the methylated ECEL1 and NT_{—}037622.5 (1393863 to 1395863) of the present invention exhibited detection rates of 55% and 85%, respectively, against liver cancer having vascular invasion and, at the same time, exhibited detection rates of 37% and 54%, respectively, against liver cancer not having vascular invasion. This means that the use of these two gene markers makes it possible to anticipate whether or not the liver cancer has vascular invasion. Here, the calculated detection rate is a ratio of the number of patients that reacted to a gene marker (a group in which methylated gene is detected) to the number of cancer patients having vascular invasion or the cancer patients not having vascular invasion. On the other hand, since the detection rates of methylated SALL3c were 45% in liver cancer having vascular invasion and 66% in liver cancer not having vascular invasion, liver cancer not having vascular invasion can be detected.

TABLE 6

| SEQ ID NO:, Accession No., | | |
|---|---|---|
| Gene Name | With vascular | Without vascular |
| (Gene Region Position) | invasion | invasion |
| SEQ ID NO: 1 | 9 subjects (45%) | 23 subjects (66%) |
| SALL3C | | |
| SEQ ID NO: 2, NM_004826 | 11 subjects (55%) | 13 subjects (37%) |
| ECEL1 | | |
| SEQ ID NO: 3, NM_001453 | 7 subjects (35%) | 8 subjects (23%) |
| FOXC1 | | |
| SEQ ID NO: 4, NM_001010848 | 4 subjects (20%) | 8 subjects (23%) |
| NRG3 | | |
| SEQ ID NO: 5, NM_014591 | 2 subjects (10%) | 5 subjects (14%) |
| KCNIP2 | | |
| SEQ ID NO: 6, NT_037622.5 | 17 subjects (85%) | 19 subjects (54%) |
| 1393863 to 1395863 | | |
| SEQ ID NO: 7, NT_022135.15 | 4 subjects (20%) | 8 subjects (23%) |
| 7774305 to 7776805 | | |
| AFP | 6 subjects (30%) | 7 subjects (20%) |
| PIVKA II | 8 subjects (40%) | 19 subjects (54%) |

### Example 3

### Reaction of Each Gene Marker Before and after Resection Operation of Liver Cancer

In the present invention, in particular, the methylated SALL3C, ECEL1, and NT_{—}037622.5 (1393863 to 1395863), which exhibit high liver cancer detection rates, were inspected before and after resection operation for whether or not the gene marker was detected. Furthermore, patient samples (specimens) were evaluated for plasma and blood cells. The results are shown by (−/−) meaning that the marker was not detected before and after operation, (+/+) meaning that the marker was detected before and after operation, (+/−) meaning that the marker was detected before operation but was not detected after the operation; and (−/+) meaning that the marker was not detected before operation but was detected after the operation. In working assumption, a disease marker that is detected before operation and not detected after operation, (+/−), is assumed to be effective. On the other hand, a marker showing (−/+) is assumed that the detection rate is low, and a marker showing (+/+), which is detected before and after operation despite of enucleation of liver cancer, is also assumed that the detection rate is low. However, since these disease markers may not be effective depending on, for example, the constitution of a patient in some cases, it is assumed that a high detection rate can be obtained compared to (−/+) and (+/+).

Then, the detection rates and non-detection rates of each gene marker before and after operation were calculated from the results of actual tests (Table 7). In Table 7, (−) means that a methylated gene was not detected (negative), and (+) means that a methylated gene was detected (positive). The results show that the methylated SALL3C, ECEL1, and NT_{—}037622.5 (1393863 to 1395863) extracted from blood cells exhibited high detection rates of 56%, 42%, and 64%, respectively, as those that are detected before operation and not detected after operation (+/−). On the other hand, these gene markers exhibited low detection rates of 20%, 36%, and 18%, respectively, as those that are also detected after operation (+/+), responding to cancer-enucleating operation. Furthermore, all of these gene markers exhibited low detection rates of 2% as those that are not detected before operation and detected after operation, but these gene markers as those that are not detected both before and after operation (−/−) exhibited low detection rates of 22%, 20%, and 16%, respectively. This shows that in liver cancer patients, the detection rates of the methylated SALL3C, ECEL1, and NT_{—}037622.5 (1393863 to 1395863) gene markers before and after operation are logical in patient groups in which these gene markers are detected, and the reliability thereof is high, though there is a group, at a certain ratio, in which the gene markers are not detected because of, for example, genetic constitution. Furthermore, the average (average of three gene markers) of the patient group in which the gene markers are not detected is 19%, but the average of the patient group in which the gene markers are detected is high, 54%, which shows that the gene markers are highly effective. In addition, DNA extracted from blood cells exhibited higher specificity and sensitivity as a test sample of a patient.

In each Example, sera and blood cells of five healthy individuals (34-year-old male, 34-year-old female, 50-year-old male, 63-year-old make, and 64-year-old female) who are known to be HBsAg (antigen of hepatitis B) negative and HCV Ab (antibody of hepatitis C) negative were similarly subjected to PCR and were thereby confirmed that methylation of the seven genes and gene regions according to the present invention was negative.

TABLE 7

| Gene Marker | Sample | −/− | +/− | +/+ | −/+ |
|---|---|---|---|---|---|
| SALL3C | plasma | 50 subjects (91%) | 4 subjects (7%) | 0 subject (0%) | 1 subject (2%) |
| SEQ ID NO: 1 | blood | 12 subjects (22%) | 31 subjects (56%) | 11 subjects (20%) | 1 subject (2%) |
| | cell | | | | |
| ECEL1 | plasma | 46 subjects (84%) | 9 subjects (16%) | 0 subject (0%) | 0 subject (0%) |
| SEQ ID NO: 2 | blood | 11 subjects (20%) | 23 subjects (42%) | 20 subjects (36%) | 1 subject (2%) |
| | cell | | | | |
| NT_037622.5 | plasma | 50 subjects (91%) | 3 subjects (5%) | 0 subject (0%) | 2 subjects (4%) |
| 1393863 to 1395863 | blood | 9 subjects (16%) | 35 subjects (64%) | 10 subjects (18%) | 1 subject (2%) |
| SEQ ID NO: 6 | cell | | | | |

1-10. (canceled)

11. A method for determining a presence or absence of a genetic factor of liver cancer, comprising detecting a methylation of a gene or gene region selected from the group consisting of SALL3c gene (SEQ ID NO:1), ECEL1 gene (SEQ ID NO:2), FOXC1 gene (SEQ ID NO:3), NRG3 gene (SEQ ID NO:4), KCNIP2 gene (SEQ ID NO:5), gene region SEQ ID NO:6, and gene region SEQ ID NO:7.

12. The method of claim 11, wherein the detecting comprises:
extracting a tissue from a subject, the tissue sample comprising a component selected from the group consisting of interstitial fluid, extravascular fluid, cerebrospinal fluid, synovial fluid, saliva, and liver tissue;
extracting a deoxyribonucleic acid (DNA) sample from the tissue;
identifying the presence of the gene or gene region in the DNA using an oligonucleotide marker comprising a component selected from the group consisting of SEQ ID NOs:8-35, wherein the marker corresponds to at least one of the genes or gene regions;
determining the presence of the methylation of the gene or gene region.

13. The method of claim 11, wherein the determining includes amplifying the identified gene or gene region through a polymerase chain reaction (PCR) using a primer that includes the oligonucleotide marker.

14. The method of claim 11, wherein the marker functions to detect the methylation of the SALL3c gene (SEQ ID NO:1), the ECEL1 gene (SEQ ID NO:2), and gene region SEQ ID NO:6.

15. The method of claim 11, wherein the method further comprises determining the frequency of the methylation in the gene or gene region.

16. The method of claim 11, wherein the method functions to detect early-stage liver cancer.

17. The method of claim 11, wherein the method functions to detect a recurrence of liver cancer.

18. A method of selecting a candidate liver cancer therapeutic agent, comprising:
selecting an agent;
culturing a mammalian cell in the presence of the agent;
culturing the mammalian cell in the absence of the agent;
detecting a methylation of a gene or gene region selected from the group consisting of SALL3c gene (SEQ ID NO:1), ECEL1 gene (SEQ ID NO:2), FOXC1 gene (SEQ ID NO:3), NRG3 gene (SEQ ID NO:4), KCNIP2 gene (SEQ ID NO:5), gene region SEQ ID NO:6, and gene region SEQ ID NO:7; and,
determining whether the agent at least suppresses the methylation of the gene or gene region; wherein, the agent is a candidate liver cancer therapeutic agent, where the agent at least suppresses the methylation.

19. A method of detecting the presence or absence of microscopic vascular invasion in liver cancer of a subject, comprising:
extracting a tissue from the subject comprising sera or blood cells;
extracting a DNA sample from the tissue; and,
detecting a methylation of a gene or gene region in the DNA, wherein the gene or gene region is selected from the group consisting of SALL3c gene (SEQ ID NO:1), ECEL1 gene (SEQ ID NO:2), and gene region SEQ ID NO:6.

20. The method of claim 19, wherein detecting the methylation of ECEL1 gene (SEQ ID NO:2) and/or gene region SEQ ID NO:6 indicates a presence of microscopic vascular invasion.

21. The method of claim 19, wherein detecting the methylation of SALL3c gene (SEQ ID NO:1) indicates an absence of microscopic vascular invasion.

22. A microarray for detecting a methylation of a gene or gene region, the microarray comprising:
a solid-phase surface;
a first marker for a SALL3c gene (SEQ ID NO:1);
a second marker for a ECEL1 gene (SEQ ID NO:2); and,
a third marker for a gene region SEQ ID NO:6;
wherein, the first, second, and third markers are immobilized on the solid phase surface for the detection of the methylation of the genes or gene region.

23. A marker for detecting a methylation of a gene or gene region selected from the group consisting of SALL3c gene (SEQ ID NO:1), ECEL1 gene (SEQ ID NO:2), FOXC1 gene (SEQ ID NO:3), NRG3 gene (SEQ ID NO:4), KCNIP2 gene (SEQ ID NO:5), gene region SEQ ID NO:6, and gene region SEQ ID NO:7, the marker comprising an oligonucleotide having a component selected from the group consisting of SEQ ID NOs:8-35.

24. The marker of claim 23 for detecting a methylation of SALL3c gene (SEQ ID NO:1) and selected from the group consisting of SEQ ID NOs:8-11.

25. The marker of claim 23 for detecting a methylation of ECEL1 gene (SEQ ID NO:2) and selected from the group consisting of SEQ ID NOs:12-15.

26. The marker of claim 23 for detecting a methylation of FOXC1 gene (SEQ ID NO:3) and selected from the group consisting of SEQ ID NOs:16-19.

27. The marker of claim 23 for detecting a methylation of NRG3 gene (SEQ ID NO:4) and selected from the group consisting of SEQ ID NOs:20-23.

28. The marker of claim 23 for detecting a methylation of KCNIP2 gene (SEQ ID NO:5) and selected from the group consisting of SEQ ID NOs:24-27.

29. The marker of claim 23 for detecting a methylation of gene region SEQ ID NO:6 and selected from the group consisting of SEQ ID NOs:28-31.

30. The marker of claim 23 for detecting a methylation of gene region SEQ ID NO:7 and selected from the group consisting of SEQ ID NOs:32-35.

31. A kit for determining a presence or absence of a genetic factor of liver cancer, comprising:
the marker of claim 23;
PCR reaction components for amplifying the gene or gene region including polymerase and an enzyme buffer;
a solid phase support; and,
a manual.
